(19) United States
(12) Patent Application Publication
(10) Pub. No.: US 2018/0236199 A1
(43) Pub. Date: Aug. 23, 2018

(54) Nasal Cannula

(71) Applicants: Kyle J. Lussier, Jacksonville Beach, FL (US); Katrina E. Lussier, Jacksonville Beach, FL (US)

(72) Inventors: Kyle J. Lussier, Jacksonville Beach, FL (US); Katrina E. Lussier, Jacksonville Beach, FL (US)

(21) Appl. No.: 15/903,165

(22) Filed: Feb. 23, 2018

Related U.S. Application Data
(60) Provisional application No. 62/462,415, filed on Feb. 23, 2017.

(51) Int. Cl. A61M 16/06; A61M 16/08

(52) U.S. Cl. CPC A61M 16/0666; A61M 16/0683; A61M 2210/0618; A61M 2205/0216; A61M 16/0875

(57) ABSTRACT

A nasal cannula having an improved nose piece, the nose piece having an elongated main body, a pair of nostril prongs and a pair of retention members bounding the nostril prongs, whereby with the nose piece applied to the nose of a patient, the nostril prongs reside within the nostrils and the retention members reside externally to the nose and serve to secure the nose piece to the nose.

This application claims the benefit of U.S. Provisional Patent Application Ser. No. 62/462,415, filed Feb. 23, 2017.

### BACKGROUND OF THE INVENTION

This application relates generally to the field of nasal cannulas used to deliver oxygen or other gases to a patient through the nostrils, and more particularly relates to nasal cannulas having a pair of tubular nasal prongs adapted for insertion into the nostrils, the tubular nasal prongs being in fluid communication with the gas supply tubing.

Nasal cannulas for supplying oxygen or other gases to patients are well known in the medical field and typically comprise an elongated tubular main body having a pair of tubular nasal or nostril prongs extending substantially perpendicularly from the main body, the combination often being referred to as a nose piece. The tubular main body is open on both ends and receives or connects with gas supply tubing or cannulas such that gas, for example oxygen, may be delivered by a compressed-gas tank, a compressor or a pump through one or both gas supply tubes into the tubular main body of the nasal cannula and then through the nostril prongs to be dispelled into the nasal passages of the patient.

To secure the device to the patient, it is common practice to position the nose piece below the nose with the nostril prongs inserted within the nostrils, wrap each of the gas supply tubes over the patient's ear and then bring the tubes together under the chin of the patient, the tubes being joined by a sliding connector such that the tubes can be tightened to snug them on the patient. Movement of the nose piece is limited to some degree by the presence of the nostril prongs in the nostrils, as the nasal prongs are positioned on each side of the centrally located columella and bounded by the inner alar walls, but dislodgement is relatively easy through turning of the head or snagging the tubes on other objects. For infants or young children, or even in certain cases with adults, it is known to address this dislodgement issue by taping the tubes to the face of the patient.

It is an object of this invention to provide an improved structure and design for a nasal cannula, and in particular for the nose piece portion of the nasal cannula, which prevent or limits accidental dislodgement, wherein the structure and design of the nasal cannula results in a more secure affixation in combination with the structure of the nose, and in a manner that is comfortable to the patient.

### BRIEF DESCRIPTION OF THE DRAWINGS

FIG. 1 is an illustration of a representative embodiment of the improved nasal cannula wherein the retention members are pre-shaped and non-formable.

FIG. 2 is an illustration of an alternative representative embodiment of the improved nasal cannula wherein the retention members are formable into different configurations.

FIG. 3 is an illustration showing an alternative embodiment of the retention members.

FIG. 4 is an illustration showing another embodiment of the retention members.

FIG. 5 is a cross-sectional view of still another embodiment of the retention members.

### SUMMARY OF THE INVENTION

In general, the invention in various embodiments is a nasal cannula comprising an elongated tubular main body, open on both ends, adapted for fluid communication with and connected to one or more gas supply tubes, a pair of tubular nostril prongs extending from and in fluid communication with the main body, each of the tubular nostril prongs being open on the non-connected end, and a pair of retention members extending from the main body, the nostril prongs being positioned between the retention members. Each retention member is spaced from the adjacent nostril prong a sufficient distance such that the retention members are positioned externally to the nose when the nostril prongs are inserted within the nostrils. The combination of the tubular main body, tubular nostril prongs and retention members defines an improved nose piece for the nasal cannula.

The retention members are thin, elongated structures, preferably composed of a plastic material. The retention members may be pre-shaped and non-formable, such that the retention members will bend or flex under force but return to their original configurations and positions when the force is removed. In this embodiment, the biasing force resulting from the mass of the nose spreading the retention members outwardly when the nasal cannula is in use maintains the nasal cannula in place. Alternatively, the retention members may be composed of a material or material combination that is formable, such that the retention members may be shaped into a desired configuration matching the configuration of the nose, and with the retention members retaining the altered configuration during use. The configured shape of the retention members maintains the nasal cannula in place due to the matching configuration alone or the matching configuration in combination with a residual biasing force smaller than the force needed to shape the retention members.

In alternative language, the invention in its various embodiments may be summarized and described as a nasal cannula comprising a nose piece and gas tubing, said nasal cannula adapted to deliver gas to a patient while mounted on the nose of a patient; said nose piece comprising a tubular main body in fluid communication with said gas tubing, two tubular, open-ended, nostril prongs extending from and in fluid communication with said main body, and two elongated retention members extending from said main body, said two nostril prongs being positioned between said two retention members; said nose piece adapted to retain said nasal cannula on a patient's nose having a central columella, two nostrils, a left and right alar wall and a left and right alar crease; whereby said nose piece is structured and adapted such that with said nose piece disposed on the patient's nose, said nostril prongs are positioned in the patient's nostrils and said retention members are positioned externally against the patient's nose. Furthermore, such invention wherein said retention members are resiliently flexible; wherein said retention members are formable; wherein the distance between each of said retention members to the nearest nostril prong is equal; wherein said retention members are biased outwardly when said nose piece is disposed on the patient's nose; wherein said retention members compress against the patient's nose when said nose piece is disposed on the patient's nose; wherein said retention members are structured and adapted to reside in the patient's alar crease when said nose piece is disposed on the patient's nose; and/or wherein each said retention member comprises an internal wire member composed of a malleable material.

Alternatively, the invention in its various embodiments may be summarized and described as a nasal cannula comprising a nose piece and gas tubing, said nasal cannula adapted to deliver gas to a patient while mounted on the nose of a patient; said nose piece comprising a tubular main body in fluid communication with said gas tubing, a left tubular, open-ended nostril prong and a right tubular, open-ended nostril prong extending from and in fluid communication with said main body, and a left elongated retention member and a right elongated retention member extending from said main body, said left and right nostril prongs being positioned between said left and right retention members; said nose piece adapted to retain said nasal cannula on a patient's nose having a central columella, two nostrils, a left and right alar wall and a left and right alar crease; wherein with said nose piece disposed on the patient's nose, said left nostril prong is adapted to be positioned to the left of the patient's columella and said right nostril prong is adapted to be positioned to the right of the patient's columella, and wherein said left retention member is adapted to be positioned to the left of the patient's left alar wall and said right retention member is adapted to be positioned to the right of the patient's right alar wall. Furthermore, such invention wherein said left retention member compresses the left alar wall and said right retention member compresses the right alar wall; wherein said left retention member resides in the left alar crease of the patient and said right retention member resides in the right alar crease; wherein said retention members are resiliently flexible; and/or wherein said retention members are formable; wherein each said retention member comprises an internal wire member composed of a malleable material.

Alternatively still, the invention in its various embodiments may be summarized and described as a nasal cannula comprising a nose piece and gas tubing, said nasal cannula adapted to deliver gas to a patient while mounted on the nose of a patient; said nose piece comprising a tubular main body in fluid communication with said gas tubing, two tubular, open-ended, nostril prongs extending from and in fluid communication with said main body, and two elongated retention members extending from said main body, said two nostril prongs being positioned between said two retention members; whereby said retention members are adapted to retain said nose piece on the nose of a patient. Furthermore, such invention wherein said retention members are resiliently flexible; wherein said retention members are formable; wherein each said retention member comprises an internal wire member composed of a malleable material.

### DETAILED DESCRIPTION OF THE INVENTION

In general, the invention in various embodiments is an improved nasal cannula that better secures the nose piece portion to the patient to prevent or limit accidental dislodgement or unwanted repositioning of the nose piece as may occur with head turning, inadvertent contact with hands or arms, or snagging on objects. The nasal cannula is also more comfortable for the patient, as less tension needs to be applied to the tubes extending around the ears to maintain the nose piece in place. Several embodiments are illustrated in the drawings, but it is to be understood that the drawings are presented for illustration and disclosure purposes and are not meant to be limiting as to the scope or definition of the invention.

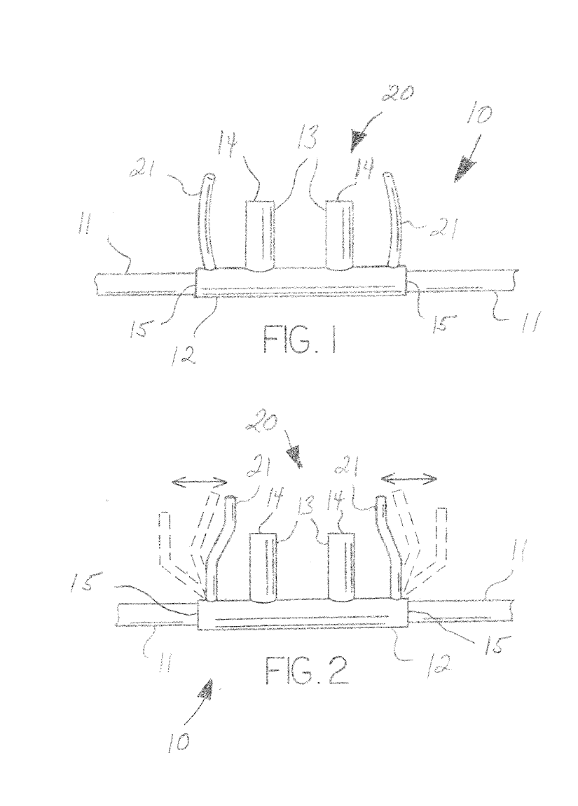

A first representative embodiment of the nasal cannula 10 is shown in FIG. 1. The nasal cannula 10 comprises an elongated tubular main body 12, having open ends 15, and a pair of tubular nostril prongs 13 in fluid communication with the main body 12, each nostril prong 13 having an open end 14 on their non-connected ends, this combination of elements defining the nose piece 20 of the nasal cannula 10. The nostril prongs 13 extend substantially perpendicularly to the longitudinal axis of the main body 12, and may be configured to be straight, curved, tapered, flared or any other commonly known configuration. The nostril prongs 13 are usually disposed equidistance from the midpoint of the main body 12 and separated a suitable distance to account for the width of the columella portion of the nose. Gas supply tubes 11, such as for example oxygen tubes, communicate with the open ends of the main body 12. The gas supply tubes 11 may be inserted into the open ends 15 of the main body 12 and retained by friction fit, adhesive bonding, or the like. The gas supply tubes 11 may be appropriately sized to themselves receive the ends of the main body 12, or the main body 12 and the tubes 11 may be manufactured as a single member. This combined structure of a tubular main body 12, open-ended nostril prongs 13 and supply tubes 11 is well known in the art. The elements are typically composed of a relatively soft, flexible, compressible, bio-compatible plastic, such as polyvinyl, latex or the like. A tank containing compressed gas, a pump or a compressor delivers gas, typically oxygen, through the gas supply tubes 11, into the main body 12 and out of the nostril prongs 13 into the nostrils of the patient.

The improvement to the nose piece 20 of the nasal cannula 10 is the provision of two retention members 21 that extend from the main body 12 in the same general direction as the nostril prongs 13, i.e., generally perpendicular to the longitudinal axis of the main body 12. The retention members 21 are separated from each other by a distance greater than the separation distance of the nostril prongs 13, and the pair of retention members 21 is disposed to the outside of and encompassing the pair of nostril prongs 13 along the main body 11, i.e., the two nostril prongs 13 are situated between the two retention members 21. Preferably the distance between a first or left retention member 21 and a first or left nostril prong 13 is the same as the distance between a second or right retention member 21 and a second or right nostril prong 13, and preferably the retention members 21 are positioned equidistance from the midpoint of the main body 12. The retention members 21 are structured and adapted to reside externally to the nostrils, as opposed to the nasal prongs which are structured and adapted to reside internally to the nostrils when the nasal cannula 10 is in use delivering gas to a patient.

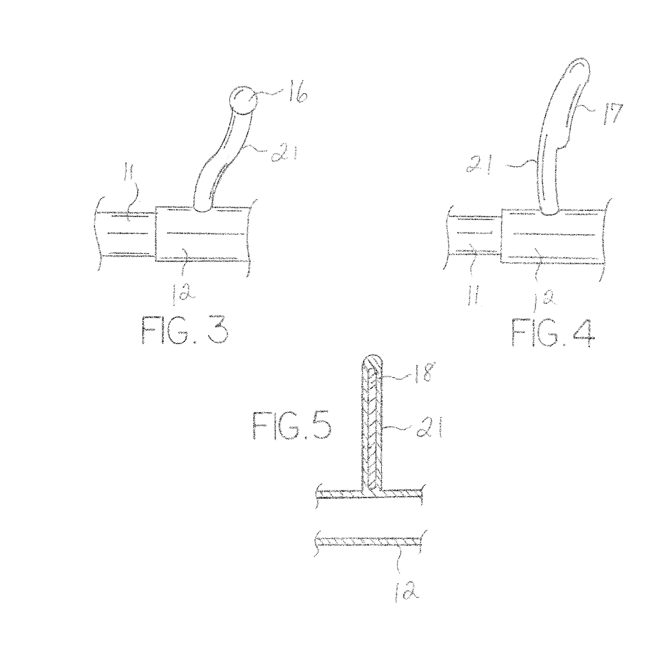

The retention members 21 as shown in the embodiment of FIG. 1 are preferably elongated, small diameter, rod-like members composed of a plastic material possessing properties whereby the retention members 21 are pre-shaped during manufacture into a desired, non-linear, in-use configuration. As illustrated, the configuration of each retention member 21 may be a slightly curved, arched or bowed shape, but other configurations that are more angular, possess inward and outward curved sections, etc., may be utilized to provide better conformity to the exterior of the nose. The material and dimensions of the retention members 21 are chosen such that the retention members 21 are resiliently flexible to the degree that allows the retention members 21 to be flexed or biased outwardly when the nasal cannula 10 is placed onto the nose, but such that the retention members 21 return to their original positioning and configuration when the expansion or biasing stress resulting from the mass of the nose situated between first retention member 21 and the first nostril prong 13 and between the second retention member 21 and second nostril prong 13 is removed. In this manner, the retention members 21 serve to mount the device onto the alar sidewalls of the nose, i.e., the outer walls of the nostrils, as the inclination of the retention members 21 is to return to their narrower non-flexed or non-biased position and configuration which conforms to the alar crease configuration. The flexing or biasing of the retention members 21 may result in actual compression of the alar wall between each retention member 21 and its corresponding nostril prong 13, which provides a more secure mounting, but the exerted pressure should not result in discomfort to the patient. Upon relief of any stress, pressure or force, the retention members 21 will return to the pre-shaped, original configuration. The configuration and placement of the pre-shaped retention members 21 may be such that the retention members 21 fit within and match the configuration of the alar crease or alar facial groove (the junction between the nose and the cheek), in which case little or no biasing force is present to maintain the nose piece 20 in proper position, the retention members 21 acting as stops or abutment members to preclude lateral movement. The length, thickness, location, separation distance, angle relative to the main body 12, etc., of the retention members 21 may vary in order to accommodate different nose sizes, etc. Enlarged ends 16, padding structures 17 or the like may also be provided on the retention members 21 for comfort or anchoring purposes, as shown in FIGS. 3 and 4.

In an alternative embodiment as illustrated in FIG. 2, the retention members 21 are composed of a formable plastic material, the term “formable” taken herein to mean a material able to be shaped by hand or tool into a desirable new configuration and orientation that does not return to its original configuration and orientation when stress or force is removed. In other words, the retention members 21 may be angled, bent, curved, etc., by hand or tool from their original configuration and orientation relative to the nostril prongs 13, with the retention members 21 retaining the new configuration and orientation when the shaping pressure or force is removed. This allows the retention members 21 to be adapted to properly fit noses of different size, shape, etc., to provide better securement and/or improved comfort. The retention members 21 may also be composed of non-formable plastic that is provided with an internal wire member 18 composed of a malleable metal, as shown in FIG. 5. As with the other embodiment of FIG. 1, the length, thickness, location, separation distance, initial angle relative to the main body, etc., of the retention members 21 may vary. Enlarged ends 16, padding structures 17 or the like may also be provided. With this structure, the retention members 21 may be shaped to provide a compressive pressure against the alar wall of the nose, the compressive pressure being less than the amount of pressure or force required to shape the retention members 21 in to a desired configuration, or the retention members 21 may be shaped to mate or conform to the configuration of the alar crease or facial grove.

It is understood that equivalents and substitutions for certain elements set forth above may be obvious to those of skill in the art, and therefore the true scope and definition of the invention is to be as set forth in the following claims.

We claim:

1. A nasal cannula comprising a nose piece and gas tubing, said nasal cannula adapted to deliver gas to a patient while mounted on the nose of a patient;
said nose piece comprising a tubular main body in fluid communication with said gas tubing, two tubular, open-ended, nostril prongs extending from and in fluid communication with said main body, and two elongated retention members extending from said main body, said two nostril prongs being positioned between said two retention members; said nose piece adapted to retain said nasal cannula on a patient's nose having a central columella, two nostrils, a left and right alar wall and a left and right alar crease;
whereby said nose piece is structured and adapted such that with said nose piece disposed on the patient's nose, said nostril prongs are positioned in the patient's nostrils and said retention members are positioned externally against the patient's nose.

2. The nasal cannula of claim 1, wherein said retention members are resiliently flexible.

3. The nasal cannula of claim 1, wherein said retention members are formable.

4. The nasal cannula of claim 1, wherein the distance between each of said retention members to the nearest nostril prong is equal.

5. The nasal cannula of claim 1, wherein said retention members are biased outwardly when said nose piece is disposed on the patient's nose.

6. The nasal cannula of claim 1, wherein said retention members compress against the patient's nose when said nose piece is disposed on the patient's nose.

7. The nasal cannula of claim 1, wherein said retention members are structured and adapted to reside in the patient's alar crease when said nose piece is disposed on the patient's nose.

8. The nasal cannula of claim 3, wherein each said retention member comprises an internal wire member composed of a malleable material.

9. A nasal cannula comprising a nose piece and gas tubing, said nasal cannula adapted to deliver gas to a patient while mounted on the nose of a patient;
said nose piece comprising a tubular main body in fluid communication with said gas tubing, a left tubular, open-ended nostril prong and a right tubular, open-ended nostril prong extending from and in fluid communication with said main body, and a left elongated retention member and a right elongated retention member extending from said main body, said left and right nostril prongs being positioned between said left and right retention members; said nose piece adapted to retain said nasal cannula on a patient's nose having a central columella, two nostrils, a left and right alar wall and a left and right alar crease;
wherein with said nose piece disposed on the patient's nose, said left nostril prong is adapted to be positioned to the left of the patient's columella and said right nostril prong is adapted to be positioned to the right of the patient's columella, and wherein said left retention member is adapted to be positioned to the left of the patient's left alar wall and said right retention member is adapted to be positioned to the right of the patient's right alar wall.

10. The nasal cannula of claim 9, wherein said left retention member compresses the left alar wall and said right retention member compresses the right alar wall.

11. The nasal cannula of claim 9, wherein said left retention member resides in the left alar crease of the patient and said right retention member resides in the right alar crease.

12. The nasal cannula of claim 9, wherein said retention members are resiliently flexible.

13. The nasal cannula of claim 9, wherein said retention members are formable.

14. The nasal cannula of claim 13, wherein each said retention member comprises an internal wire member composed of a malleable material.

15. A nasal cannula comprising a nose piece and gas tubing, said nasal cannula adapted to deliver gas to a patient while mounted on the nose of a patient;
said nose piece comprising a tubular main body in fluid communication with said gas tubing, two tubular, open-ended, nostril prongs extending from and in fluid communication with said main body, and two elongated retention members extending from said main body, said two nostril prongs being positioned between said two retention members;
whereby said retention members are adapted to retain said nose piece on the nose of a patient.

16. The nasal cannula of claim 15, wherein said retention members are resiliently flexible.

17. The nasal cannula of claim 15, wherein said retention members are formable.

18. The nasal cannula of claim 17, wherein each said retention member comprises an internal wire member composed of a malleable material.
